(19) United States
(12) Patent Application Publication
(10) Pub. No.: US 2023/0080394 A1
(43) Pub. Date: Mar. 16, 2023

(54) METHODS OF PRODUCING SOMATIC HYBRID AND CYBRID ORGANISMS

(71) Applicant: Blue Sun Mycology Group, LLC, Lebanon, OR (US)

(72) Inventor: Jerred Tudela, Lebanon, OR (US)

(21) Appl. No.: 17/945,814

(22) Filed: Sep. 15, 2022

Related U.S. Application Data
(60) Provisional application No. 63/244,445, filed on Sep. 15, 2021.

(51) Int. Cl. C12N 15/02; C12N 15/04; C12N 1/14; A01G 18/20; C12R 1/645

(52) U.S. Cl. CPC C12N 15/02; C12N 15/04; C12N 1/145; A01G 18/20; C12R 2001/645

(57) ABSTRACT

Methods of producing a hybrid organism. The methods include providing a first organism, providing a second organism, providing a fusing medium, combining the first organism and the second organism in or on the fusing medium to define a fusion environment, and initiating somatic fusion between the first organism and the second organism in the fusion environment to produce the hybrid organism. The first organism is either a plant organism or a fungus organism. The second organism is either a plant organism or a fungus organism. In some examples, the first organism is Tuber melanosporum . In certain examples, the second organism is either Panaeolus cambodginiensis or Panaeolus cyanescens . In some examples, the methods include incubating the hybrid organism, regenerating cell walls of the hybrid organism, and/or replicating the hybrid organism.

### CROSS REFERENCE TO RELATED APPLICATIONS

This application claims priority to copending U.S. Application, Ser. No. 63/244,445, filed on Sep. 15, 2021, which is hereby incorporated by reference for all purposes.

### BACKGROUND

The present disclosure relates generally to methods of cultivating fungi and plant organisms. In particular, methods of producing somatic hybrid and cybrid fungi and plant organisms are described.

Sexual hybridization has been the conventional method for improving the characteristics in cultivated plants and fungi for years. The major limitation to sexual hybridization is that it can only be performed within a particular plant or fungi species or between closely related species. Sexual hybridization being limited to interspecies or closely related species applications reduces the chances of significantly improving a plant or fungi species via sexual hybridization.

In some instances, species barriers can be overcome by somatic cell fusion. Somatic hybridization involves fusing isolated protoplasts in vitro to form a hybrid cell and developing the hybrid cell into a hybrid plant or fungus. Somatic cell fusion can form viable hybrids and cybrids (hereinafter often collectively just “hybrids” for simplicity) not possible through sexual hybridization.

As an example, somatic hybridization has been used to form a patentable species of pet known as a GloFish. The GloFish was formed by fusing green fluorescent protein (GFP) protoplasts with zebrafish embryo protoplasts. GFP was originally extracted from a jellyfish and naturally produces bright green fluorescence. The new GloFish hybrid has the characteristics of both GFP and zebrafish.

Somatic hybridization has opened new possibilities for genetically manipulating plants and fungi in vitro to improve crops. Some of the practical applications for somatic hybridization include:

Disease Resistance

Somatic hybridization has helped to transfer disease resistant genes from one species to another. For example, resistance against TMV, spotted wilt virus, and insect pests has been introduced in tomatoes using somatic hybridization.

Environmental Tolerance

Environmental factors, such as cold, frost, and salt, pose a challenge for crops. Somatic hybridization has successfully introduced tolerance to environmental factors into crops. For example, somatic hybridization has introduced increased cold tolerance into tomatoes.

Quality Characteristics

Somatic hybridization has successfully increased certain quality characteristics of plants. For example, somantic hybridization has been used to increase the nicotine content of tobacco plants and to reduce the erucic acid concentration of rapeseed.

Thus, somatic hybridization methods have compelling potential to produce hybrid and cybrid fungi and plant organisms with unique and useful characteristics and properties. Examples of new and useful somatic hybridization methods are discussed below.

Disclosure relevant to somatic hybridization methods is provided in U.S. Pat. No. 4,996,390. The complete disclosure of this patent is herein incorporated by reference for all purposes.

### SUMMARY

The present disclosure is directed to methods of producing a hybrid organism. The methods include providing a first organism, providing a second organism, providing a fusing medium, combining the first organism and the second organism in or on the fusing medium to define a fusion environment, and initiating somatic fusion between the first organism and the second organism in the fusion environment to produce the hybrid organism.

The first organism is either a plant organism or a fungus organism. The second organism is either a plant organism or a fungus organism.

In some examples, the first organism is Tuber melanosporum . In certain examples, the second organism is either Panaeolus cambodiginiensis or Panaeolus cyanescens . In some examples, the methods include incubating the hybrid organism, regenerating cell walls of the hybrid organism, and/or replicating the hybrid organism.

### BRIEF DESCRIPTION OF THE DRAWINGS

FIG. 1 is a schematic diagram of a first somatic hybridization method.

FIG. 2 is a schematic diagram of a second somatic hybridization method.

FIG. 3 is a schematic diagram of a third somatic hybridization method.

### DETAILED DESCRIPTION

The disclosed somatic hybridization methods will become better understood through review of the following detailed description in conjunction with the figures. The detailed description and figures provide merely examples of the various inventions described herein. Those skilled the art will understand that the disclosed examples may be varied, modified, and altered without departing from the scope of the inventions described herein. Many variations are contemplated for different applications and design considerations; however, for the sake of brevity, each an every contemplated variation is not individually described in the following detailed description.

Throughout the following detailed description, a variety of somatic hybridization method examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Spore-producing mushrooms” means any normal, spore producing, physical expression type fruit body belonging to the fungi genus Psilocybe or Panaeolus.

“Sporeless mushrooms” means any normal or abnormal physical expression type fruit body belonging to the fungi genera Psilocybe or Panaeolus that lacks the ability to produce spores.

“Abnormal mutations” means any fruit body that shows any physical expression not normal to Psilocybe or Panaeolus fungi. Such mutations may include, for example, blob type mutations, fin type mutations, coral type mutations, or any fungi with mutated caps or bodies.

“Sclerotia” means the underground growing, truffle type fruit bodies that certain Psilocybe or Panaeolus species are capable of producing.

“Monokaryotic” means hyphae and mycelium that contain nuclei of one same genotype. Monokaryotic is interchangeable with heterokaryotic, homokaryotic, and uninucleate.

“Dikaryotic” means mycelium that contain binucleate cells.

“Binucleate” means cells that contain two nuclei.

“Hyphal anastomosis” means cellular fusion between branches of the same or different hyphae or mycelium.

“Hyphae” means individual cellular threads that form when spores germinate.

“Mycelium” means a collection or grouping of hyphae that have formed ropelike threads creating a web-like network.

“Culture” means a product of the cultivation of a living microorganism on a prepared nutrient medium. Such a microorganism may include, for example, Psilocybe or Panaeolus mycelium.

“Sub-variant” means a subsidiary variant or subtype of a Psilocybe or Panaeolus species. Such subtypes, for example, may include a wild Psilocybe or Panaeolus species collected from a certain location or isolated phenotypes of domesticated or wild Psilocybe or Panaeolus species.

“Domesticated species” means a Psilocybe or Panaeolus species that has been stabilized from generational selection and line breeding.

“Wild species” means a Psilocybe or Panaeolus species that has been collected from its natural growing habitat.

“Line breeding” means a form of inbreeding that involves making selections and collecting and growing spores from those selections. The inbreeding process is repeated through multiple generations so that only one or few phenotypes occur more than once within a Psilocybe or Panaeolus species sub-variant.

“Vegetatively compatible” means compatible to mate in the vegetative or non-fruiting stage of growth.

Methods of Producing Somatic Hybrid and Cybrid Organisms

With reference to the figures, methods of producing somatic hybrid and cybrid organisms will now be described. The methods described herein function to bioengineer intergeneric hybrid organisms by fusing protoplasts of distinct organisms, recovering a hybrid organism, and regenerating cell walls of the hybrid organism cells. The organisms may be fungi organisms or plant organisms and the intergeneric hybrid organism produced may be a hybrid plant, a hybrid fungi or combination of both.

In one specific example, the novel methods described herein were used to produce novel intergeneric hybrids of Tuber melanosporum and Panaeolus cambodginiensis. Tuber melansporum is commonly referred to as “Black Truffle” and Panaeolus cambodginiensis is commonly referred to as “Goliath.”

In another specific example, the novel methods described herein were used to produce novel intergeneric hybrids of Tuber melansporum and Panaeolus cyanescens. Panaeolus cyanescens is commonly referred to as “Jamaica.”

### Somatic Hybrid Method Embodiment One

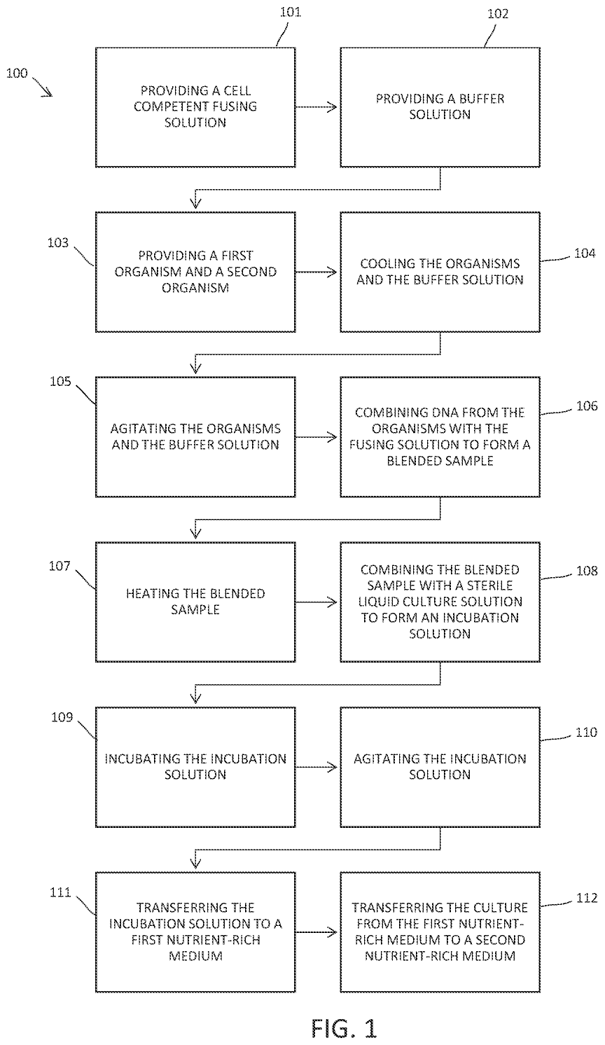

With reference to FIG. 1, a first example of a somatic hybrid method, method 100, will now be described. Method 100 includes providing a cell competent fusing solution at step 101 and providing a buffer solution at step 102. At step 103, method 100 includes providing a first organism and a second organism. At step 104, method 100 includes cooling the first organism, the second organism, and the buffer solution.

At step 105, method 100 includes separately agitating the first organism, the second organism, and the buffer solution. Step 106 of method 100 includes combining DNA from the first organism and the second organism with the fusing solution to form a blended sample. Method 100 includes heating the blended sample at an optional step 107. At step 108, method 100 includes combining the blended sample a sterile liquid culture solution to form an incubation solution.

Method 100 proceeds with incubating the incubation solution at step 109. At step 110, method 100 includes agitating the incubation solution while it incubates. Method 100 further includes transferring the incubation solution to a first nutrient-rich medium to regenerate cell walls at step 111. At step 112, method 100 includes transferring the culture from the first nutrient-rich medium to a second nutrient-rich medium.

In some examples, the somatic hybrid methods do not include one or more steps included in method 100. For example, some method examples do not include heating the blended sample or transferring the culture from the first nutrient-rich medium to a second nutrient-rich medium. In other examples, the method includes additional or alternative steps.

Providing a Fusing Solution

Providing a fusing solution at step 101 may include preparing a using solution or sourcing an already prepared fusing solution. The fusing solution is selected to be cell competent in view of the first organism and the second organism. The fusing solution may be any currently known or later developed cell competent fusing solution suitable for the organisms selected for the somatic hybrid method and the particular somatic hybrid method conditions.

Providing a Buffer Solution

Providing a buffering solution at step 102 may include preparing a buffering solution or sourcing an already prepared buffering solution. The buffering solution may be any currently known or later developed buffering solution suitable for the organisms selected for the somatic hybrid method and the particular somatic hybrid method conditions.

In one example of step 102, the buffer solution includes competent cell culture; 10% polyethylene glycol; 5% dimethyl sulfoxide (DMSO); and 25 mM calcium chloride (CaCl2). The competent cell culture may be E. coli. The polyethylene glycol may have a molecular weight of 3350 or 8000.

Providing First and Second Organisms

Providing first and second organisms at step 103 establishes the genetic material that will be donated and somatically fused in the somatic hybrid method. A wide variety of organisms may be provided, include plant organisms, fungi organisms, and combinations thereof.

In one specific example, the first organism is Tuber melanosporum. Tuber melansporum is commonly referred to as “Black Truffle.”

In some examples where the first organism is Tuber melanosporum the second organism is Panaeolus cambodginiensis. Panaeolus cambodginiensis is commonly referred to as “Goliath.” In another example where the first organism is Tuber melanosporum , the second organism is Panaeolus cyanescens. Panaeolus cyanescens is commonly referred to as “Jamaica.”

Cooling the Organisms and the Buffer Solution

Cooling the organisms and the buffering solution at step 104 prepares the organisms to undergo subsequent protoplast fusion processing steps. In some examples, the buffer solution and the donor DNA samples are cooled by placing them on ice for 15 minutes. Longer, shorter, and different cooling methods may be used.

Agitating the Organisms and the Buffer Solution

Agitating the organisms and the buffering solution at step 105 integrates the organisms with the buffering solution. Integrating the organisms with the buffering solution attenuates pH changes in the organisms and in the environment surrounding the organisms during the somatic hybrid method.

In some examples, agitation is accomplished by shaking the buffer solution and DNA samples well for at least 30 seconds by hand. Alternatively, a vortex mixer may be used.

Combining DNA from Each Organism with the Fusing Solution

Combining DNA from the organisms with the fusion solution at step 106 initiates somatic fusion, also known as protoplast fusion, of the two organisms. In one example, the combination step includes pipetting 0.5 ul of the fusion solution into a clean microcentrifuge tube. Further in that particular example, the combination step includes pipetting 0.5 ul of DNA from each genetic donor organism into the same tube with the fusion solution.

Heating the Blended Sample

Heating the blended sample at step 107 is an optional step. Some organisms do not require heating. For organisms that do not require heating, the heating steps described herein can be skipped.

In the present example, heating the blended sample at step 107 includes heating the blended sample in a heat bath at 99 degrees F. for 60 mins.

Combining the Blended Sample with a Sterile Liquid Culture Solution

Combining the blended sample with a sterile liquid culture solution at step 108 forms an incubation solution. In one example of step 108, the blended sample is transferred into a 15 ml centrifuge tube. Further, 8 ml of a premixed and sterile liquid culture solution is added into the 15 ml centrifuge tube with the blended sample.

The sterile liquid culture solution may be prepared as part of the somatic hybrid method or sourced beforehand. The sterile liquid culture solution may be any sterile liquid culture solution currently existing or later developed suitable for the organisms involved.

Incubating the Incubation Solution

Incubating the incubation solution at step 109 enables protoplasts of the first and second organisms to fuse. In the example shown in FIG. 1, incubating the incubation solution at step 109 includes holding the incubation solution in a 15 ml centrifuge tube at 86 degrees F. for 3 to 7 days. In other examples, the incubation solution is held at warmer or cooler temperatures. The incubation time may be varied as needed for a given combination of organisms.

Agitating the Incubation Solution

In the example shown in FIG. 1, incubating the incubation solution at step 110 includes occasionally agitating the incubation solution as it incubates. Further, incubating the incubation solution at step 110 includes vigorously agitating the incubation solution after the incubation step 109 is complete.

The incubation solution may be agitated with a vortex mixer. Additionally or alternatively, the incubation solution may be agitated by manually shaking the vessel containing the incubation solution.

Transferring the Incubation Solution to a First Nutrient-Rich Medium

Transferring the incubation solution to a first nutrient-rich medium at step 110 functions to regenerate cell walls in the hybrid organism cells. The first nutrient-rich medium may be agar or any other suitable nutrient-rich medium.

Transferring the Culture to a Second Nutrient-Rich Medium

Transferring the culture from the first nutrient-rich medium to a second nutrient-rich medium at step 112 functions to replicate the hybrid organism formed in the somatic hybrid method. In the example shown in FIG. 1, transferring the culture to the second nutrient-rich medium at step 112 occurs after cell walls in the hybrid organism cells have regenerated. The second nutrient-rich medium may be agar or any other suitable nutrient-rich medium.

### Additional Embodiments

The discussion will now focus on additional somatic hybrid method embodiments. The additional embodiments include many similar or identical features to method 100. Thus, for the sake of brevity, each feature of the additional embodiments below will not be redundantly explained. Rather, key distinctions between the additional embodiments and method 100 will be described in detail and the reader should reference the discussion above for features substantially similar between the different method examples.

### Somatic Hybrid Method Embodiment Two

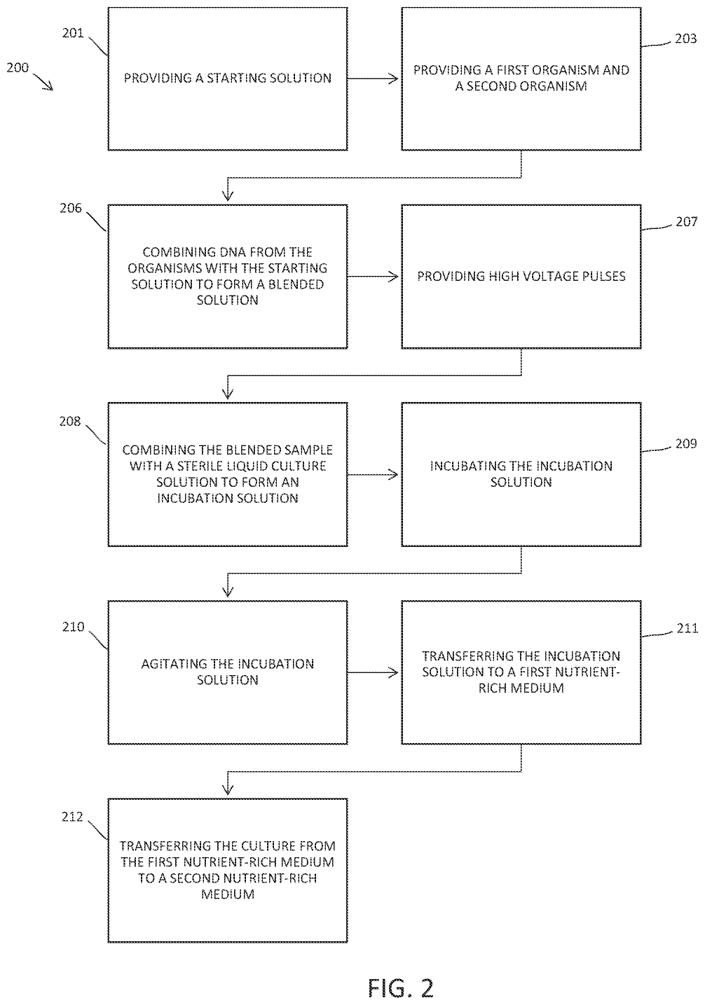

Turning attention to FIG. 2, a second example of a somatic hybrid method, somatic hybrid method 200, will now be described. A distinction between method 200 and method 100 is that method 200 utilizes electroporation to cause nearby cells to undergo electrofusion when subjected to high voltage pulses.

As can be seen in FIG. 2, method 200 includes providing a starting solution at step 201. At step 203, method 200 includes providing a first organism and a second organism.

Step 206 of method 200 includes combining DNA from the first organism and the second organism with the starting solution to form a blended sample. Method 200 includes providing high voltage pulses to the blended sample at step 207. At step 208, method 200 includes combining the blended sample with a sterile liquid culture solution to form an incubation solution.

Method 200 proceeds with incubating the incubation solution at step 209. At step 210, method 200 includes agitating the incubation solution while it incubates. Method 200 further includes transferring the incubation solution to a first nutrient-rich medium to regenerate cell walls at step 211. At step 212, method 200 includes transferring the culture from the first nutrient-rich medium to a second nutrient-rich medium.

Providing a Starting Solution

Providing a starting solution at step 201 enables creating an environment conducive to fusing the first and second organisms in steps 206-210. The starting solution may be a cell storage solution or an electrofusion solution. The cell storage solution may be any currently known or later developed cell storage solution compatible with the first and second organisms. Similarly, the electrofusion solution may be any currently known or later developed electrofusion solution compatible with the first and second organisms.

Providing High Voltage Pulses

Providing high voltage pulses to the blended sample at step 207 initiates electrofusion between nearby cells of the first organism and the second organism. In the example shown in FIG. 2, providing high voltage pulses to the blended sample at step 207 includes providing six pulses of 0.71 ms at an estimated cell membrane voltage of about 450 mV. In some examples, fewer than six pulses are provided to the blended sample. In certain examples, more than six pulses are provided to the blended sample.

The duration of the pulses may be varied. For example, the pulses may be shorter or longer than 0.71 ms.

The voltage of the pulses may also be varied. In some examples, the voltage of the pulses is less than 450 mV. In other examples, the voltage of the pulses is greater than 450 mV. Voltages conducive with the membrane of the first and second organisms have been observed to be most effective at initiating electrofusion.

### Somatic Hybrid Method Embodiment Three

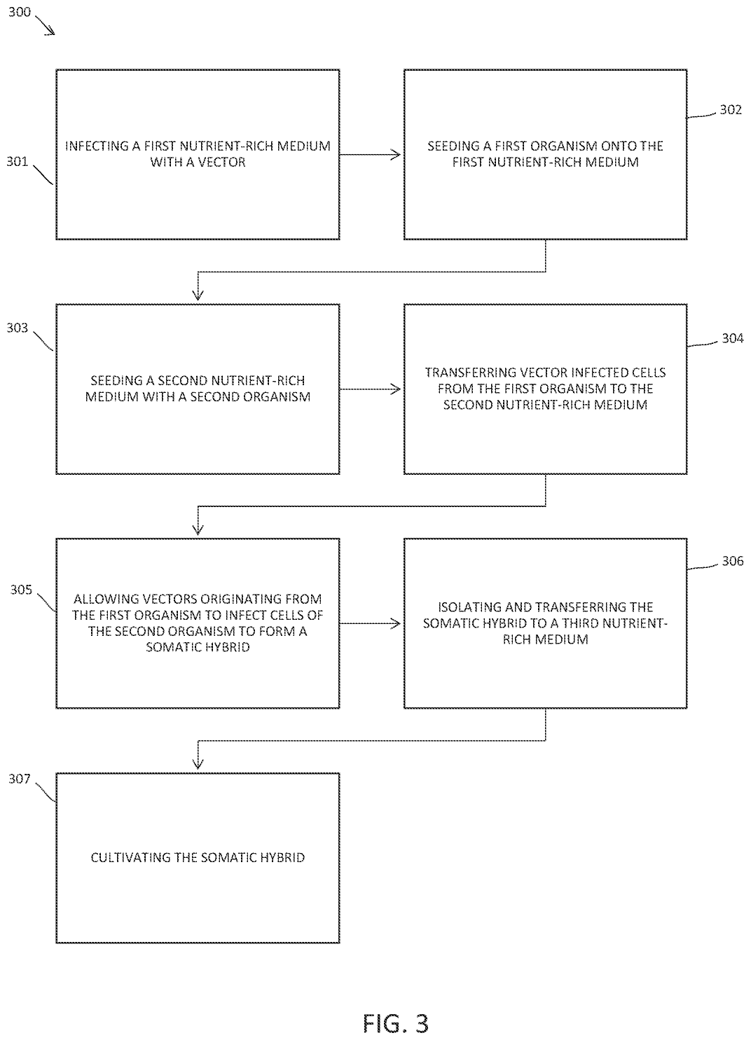

Turning attention to FIG. 3, a third example of a somatic hybrid method, somatic hybrid method 300, will now be described. A distinction between method 300 and method 100 is that method 300 utilizes vectors to transfer genetic material between organisms.

Method 300 includes infecting a first nutrient-rich medium with a vector at step 301. At step 302, a first organism is seeded onto the first nutrient-rich medium infected with the vector and allowed to replicate. Method 300 farther includes seeding a second nutrient-rich medium with a second organism at step 303.

At step 304, sector infected cells from the first organism on the first nutrient-rich medium are transferred to the second nutrient-rich medium. Method 300 continues with allowing vectors originating from the first organism to infect cells of the second organism on the second nutrient-rich medium at step 305 to form a somatic hybrid. At step 306, the somatic hybrid is isolated and transferred to a third nutrient-rich medium. Method 300 concludes with cultivating the somatic hybrid at step 307.

Infecting a First Nutrient-Rich Medium with a Vector

Infecting a first nutrient-rich medium with a vector at step 301 establishes a carrier for transferring genetic material from the first organism to the second organism in steps 304 and 305. The vector may be any currently known or later discovered or developed vector suitable for the first organism and the second organism. In some examples, the vector is Agrobacterium and E. coli. The vector may be any virus, yeast, mold, or bacteria with transformative gene properties.

Seeding the First Nutrient-Rich Medium with the First Organism

Seeding the first nutrient-rich medium with the first organism at step 302 brings the vector and first organism into contact with each other. When the first organism is in contact with the vector, the vector infects the first organism. The vector collects genetic material from the first organism when it infects the first organism.

Allowing Vector to Infect Cells of the Second Organism

Allowing vectors originating from the first organism to infect the second organism at step 305 transfers genetic material from the first organism to the second organism. Transferring genetic material from the first organism to the second organism with the vector established in step 301 forms a somatic hybrid organism. In step 305, the vector serves as a carrier of genetic material between the first organism and the second organism.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, nether requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A method of producing a hybrid organism, the method comprising the steps of:
providing a first organism, where the first organism is either a plant organism or a fungus organism;
providing a second organism, where the second organism is either a plant organism or a fungus organism;
providing a fusing medium;
combining the first organism and the second organism in or on the fusing medium to define a fusion environment; and
initiating somatic fusion between the first organism and the second organism in the fusion environment to produce the hybrid organism.

2. The method of claim 1, further comprising incubating the hybrid organism.

3. The method of claim 2, wherein incubating the hybrid organism includes:
adding a sterile solution to the fusion environment to define an incubation solution; and
holding the incubation solution within a selected incubation temperature range for a selected incubation duration.

4. The method of claim 3, wherein incubating the hybrid organism further includes agitating the incubation solution.

5. The method of claim 1, further comprising regenerating cell walls of the hybrid organism.

6. The method of claim 5, wherein regenerating cell walls of the hybrid organism includes transferring the hybrid organism to a nutrient-rich medium and maintaining the hybrid organism in or on the nutrient-rich medium for a selected regeneration duration.

7. The method of claim 6, further comprising replicating the hybrid organism.

8. The method of claim 7, wherein:
the nutrient-rich medium is a first nutrient-rich medium; and
replicating the hybrid organism includes transferring the hybrid organism from the first nutrient-rich medium to a second nutrient rich medium and maintaining the hybrid organism in or on the second nutrient-rich medium for a selected replication duration.

9. The method of claim 1, wherein the fusing medium is a fusing solution.

10. The method of claim 1, wherein the fusing medium is a cell storage solution.

11. The method of claim 1, wherein the fusing medium is an electrofusion solution.

12. The method of claim 1, wherein the fusing medium is a nutrient-rich growth medium.

13. The method of claim 1, wherein initiating somatic fusion includes heating the fusion environment to a selected fusion temperature range for a selected fusion duration.

14. The method of claim 1, wherein initiating somatic fusion includes providing high-voltage electric pulses to the fusion environment.

15. The method of claim 1, wherein:
the method further comprises infecting the first organism with a vector to define a vector infected first organism;
combining the first organism and the second organism in or on the fusing medium to define a fusion environment includes combining the vector infected first organism and the second organism in or on the fusing medium; and
initiating somatic fusion includes holding the vector infected first organism and the second organism in or on the fusing medium for a selected fusion duration to allow the vector to transfer genetic material from the vector infected first organism to the second organism.

16. The method of claim 15, wherein the vector includes Agrobacterium tumefaciens.

17. The method of claim 1, wherein the first organism is Tuber melanosporum.

18. The method of claim 17, wherein the second organism is Panaeolus cambodginiensis.

19. The method of claim 17, wherein the second organism is Panaeolus cyanescens.

20. A method of producing a hybrid organism, the method comprising the steps of:
providing a first organism, where the first organism is Tuber melanosporum;
providing a second organism, where the second organism is either Panaeolus cambodginiensis or Panaeolus cyanescens;
providing a fusing medium;
combining the first organism and the second organism in or on the fusing medium to define a fusion environment;
initiating somatic fusion between the first organism and the second organism in the fusion environment to produce the hybrid organism;
incubating the hybrid organism;
regenerating cell walls of the hybrid organism; and
replicating the hybrid organism.
